(19) United States
(12) Patent Application Publication
(10) Pub. No.: US 2012/0122235 A1
(43) Pub. Date: May 17, 2012

(54) Devices and Methods for Detection of Biomolecular Interactions

(75) Inventors: P. Bryant Chase, Tallahassee, FL (US); Khaled Aledealat, Tallahassee, FL (US); Kan-Sheng Chen, Westmont, IL (US); Steven M. Hira, Atlanta, GA (US); Geoffrey F. Strouse, Tallahassee, FL (US); Stephan von Molnar, Tallahassee, FL (US); Peng Xiong, Tallahassee, FL (US)

(73) Assignee: FLORIDA STATE UNIVERSITY RESEARCH FOUNDATION, INC., Tallahassee, FL (US)

(21) Appl. No.: 13/276,193

(22) Filed: Oct. 18, 2011

Related U.S. Application Data
(60) Provisional application No. 61/393,981, filed on Oct. 18, 2010.

(51) Int. Cl. G01N 33/53; G01N 27/00; B82Y 15/00

(52) U.S. Cl. 436/501; 422/82.01; 977/773

(57) ABSTRACT

Devices, systems, and methods are provided for the detection of biomolecular interactions. The interactions between one or more target DNA strands, one or more receptor DNA strands, and one or more probe DNA strands, if necessary, are used to detect the one or more target DNA strands. The one or more target DNA strands or the one or more probe DNA strands may be coupled to a magnetic bead, and the one or more receptor strands may be coupled to the Hall device.

### CROSS-REFERENCE TO RELATED APPLICATION

This application claims priority to U.S. Provisional Application No. 61/393,981, filed Oct. 18, 2010, which is incorporated herein by reference.

### STATEMENT REGARDING FEDERALLY SPONSORED RESEARCH OR DEVELOPMENT

This invention was made with government support under grant number R21 GM079592 (PBC) awarded by the National Institute of Health and National Institute of General Medical Sciences. The government has certain rights to this invention.

### FIELD OF THE INVENTION

This invention relates generally to devices for detecting biomolecular interactions, and methods for detecting biomolecular interactions.

### BACKGROUND

Rapid detection of target nucleic acids at the genomic level in a biological matrix is a critical technological hurdle for the development of a practical point of care (POC) device (Giljohann, D. A., et al. NATURE 462, 2009, 461-464; Ince, J. et al., EXPERT REVIEW OF MEDICAL DEVICES 6, 2009, 641-651).

Bio-sensors based on flow-cytometry (Song, X. D. et al. ANAL. BIOCHEM. 284, 2000, 35-41), optical chip/microarrays (Shi, L. M., et al. NATURE BIOTECHNOLOGY 24, 2006, 1151-1161; Johnson, D. S., et al. GENOME RESEARCH 18, 2008, 393-403), Raman spectroscopy (Cao. Y. W. C., et al., SCIENCE 297, 2002, 1536-1540), surface plasmon resonance (SPR) spectroscopy (Campbell, C. T. et al., BIOMATERIALS 28, 2007, 2380-2392), colorimetric assays (Taton, T. A., et al. SCIENCE 289, 2000, 1757-1760; Hill, H. D. et al., ANAL. CHEM. 79, 2007, 9218-9223; Festag, G. et al., J. or FLUORESCENCE 15, 2005, 161-170), energy transfer assays (Dubertret, B., et al., NATURE BIOTECHNOLOGY 19, 2001, 365-370; Algar, W. R. et al., TRAC-TRENDS IN ANALYTICAL CHEMISTRY 28, 2009.292-306; Algar, W. R. et al., ANALYTICAL CHEMISTRY 81, 2009, 4113-4120), nanomechanical (Husale. S. et al., NATURE 462, 2009, 1075-1138) and electrostatic readout devices (Clack, N. G. et al., NATURE BIOTECHNOLOGY 26, 2008, 825-830) suffer from various limitations in stability, portability, sensitivity, and selectivity.

Optical microarrays offer the ability to multiplex, yet are often not label-free, are instrumentally intensive, and can be cost prohibitive to employ at the POC level. Magnetic based sensing technologies offer an alternative bio-sensor platform that is compatible with a plethora of biological environments, exhibit low biological background, can be mass produced, and if configured properly, can offer dynamic, label-free detection in a micro-fabricated scalable platform (Arruda, D. G. et al EXPERT REVIEW OF MOLECULAR DIAGNOSTICS 9, 2009, 749-755; Schotter, J. et al., BIOSENSORS & BIOELECTRONICS 19, 2004, 1149-1156). The magnetic transduction technology includes giant magnetoresistive (GMR) based sensors including both spin valves (Martins, V. C. et al., Biosensors & Bioelectronics 24, 2009, 2690-2695; Germano, J. et al., Sensors 9, 2009, 4119-4137; Graham, D. L. et al., SENSORS AND ACTUATORS B-CHEMICAL 107, 2005, 939-944; Ferreira, H. A. et al., IEEE TRANSACTIONS ON MAGNETICS 41, 2005, 4140-4142; Ferreira, H. A. et al., J. OF APPLIED PHYSICS 99, 2006; Hall, D. A. et al., BIOSENSORS & BIOELECTRONICS 24, 2051-2057) and bead array counters (BARC) (Baselt, D. R. et al., BIOSENSORS & BIOELECTRONICS 13, 1998, 731-739; Edelstein, R. L. et al., BIOSENSORS & BIOELECTRONICS 14 2000, 805-813) and Hall based sensors (Togawa, K. et al. JAPANESE JOURNAL OF APPLIED PHYSICS PART 2-LETTERS AND EXPRESS LETTERS 44, 2005, L1494-L1497; Sandhu, A. et al., BIOSENSORS & BIOELECTRONICS 22, 2007, 2115-2120; Mihajlovic, G. et al., APPLIED PHYSICS LETTERS 87, 2005; Jihajlovic,G. et al., JOURNAL OF APPLIED PHYSICS 102, 2007; Manandhar, P. et al., NANOTECHNOLOGY 20, 2009). Magnetic based sensors have demonstrated detection of matrix-insensitive proteins at the fM level using a GMR device (Gaster, R. S. et al., NATURE MEDICINE 15, 2009, 1327-U1130).

Present technologies for nucleic acid hybridization are “gene chips” by companies such as Affymetrix, Inc. and. Roche NimbleGen, Inc. These technologies are not “label-free” and require “amplification” of the nucleic acids of interest for detection, i.e., the nucleic acid must be replicated through biochemical, enzymatic synthesis and adding a fluorescent label for detection. There arc a number of assays for protein detection, the closest being “ELISA” immunoassays. All of these existing assays report via an optical signal.

Therefore, devices and methods are needed that do not require the replication of DNA samples, which can introduce errors at a low, but not nil, frequency. Devices and methods are also needed that are “label-free,”—i.e., there is no need to modify the molecule of interest when the assay is properly configured. Devices and methods are also needed that provide an electrical signal via micro-Hall magnetometry, while providing an optional redundant optical signal. Since optics are typically bulky, devices and methods are also needed that are more portable and easier to directly interface with actuator/response devices. An improved device having at least one of these features would be desirable.

### BRIEF SUMMARY

Devices, systems, and methods are provided for the detection of biomolecular interactions. In one aspect, devices or systems are provided comprising a Hall device, one or more receptor DNA strands pre-assembled on the Hall device surface, a magnetic head, and one or more probe DNA strands pre-conjugated to the magnetic bead. In these devices or systems, the one or more receptor DNA strands and one or more probe DNA strands are configured to interact with one or more target DNA strands via complementary base pairing.

In another aspect, devices or systems are provided comprising a Hall device, one or more receptor DNA strands pre-assembled on the Flail device surface, a magnetic bead, and one or more target DNA strands pre-conjugated to the magnetic bead. In these devices or systems, the one or more receptor DNA strands are configured to interact with the one or more target DNA strands via complementary base pairing.

In a further aspect, methods are provided comprising pre-assembling one or more receptor DNA strands on a surface of a flail device, pre-conjugating one or more probe DNA strands to a magnetic bead, and detecting the coupling of the magnetic bead to the Hall device. In these methods, the one or more receptor DNA strands and the one or more probe DNA strands are configured to interact with one or more target DNA strands via complementary base pairing.

In a still further aspect, methods are provided comprising pre-conjugating one or more target DNA strands to a magnetic bead, pre-assembling one or more receptor DNA strands on a surface of a Hall device, and detecting the coupling of the magnetic bead to the Hall device. In these methods, the one or more receptor DNA strands are configured to interact with the one or more target DNA strands via complementary base pairing.

### BRIEF DESCRIPTION OF THE DRAWINGS

FIG. 1A depicts a generalized schematic for the detection of label-free target DNA using Hall magnetometry.

FIG. 1B depicts the optical microscopy characterization (wide-field fluorescence and DIC overlay) of three-strand DNA assembly.

FIG. 1C depicts Hall responses for three active junctions (i, ii, iii) and a single control junction (iie).

FIG. 1D depicts the SEM image used to evaluate the location and quantify the number of nanobeads contributing to the Hall response for (iii).

FIG. 1E depicts the theoretical device signal stemming from a single 344 nm SPM bead as a function of position over the Hall junction.

FIG. 2A a three-strand DNA assembly on a mimic array (patterned on a GaAs substrate) for complementary target only.

FIG. 2B depicts a line scan of the wide-field fluorescence microscopy image in FIG. 2A.

FIG. 2C depicts a three-strand DNA assembly on a mimic array (patterned on a GaAs substrate) for a 10 ppm target in non-target DNA.

FIG. 3A is a pictorial representation of two-strand DNA assembly, wherein the probe DNA is complementary to the receptor DNA.

FIG. 3B depicts Hall response data for the active (iv) and control Hall junction (ive) plotted as Hall voltage versus time, where the drop in Hall voltage corresponds to the presence of a magnetic nanobead.

FIG. 3C depicts an SEM image which confirms only one nanobead contributed significantly to the signal measured in (iv).

FIG. 3D depicts the theoretical Hall device cross-sectional response for a single 344 nm SPM (super paramagnetic) bead as a function of position from the center of the junction.

FIG. 4A shows an SEM micrograph depicting label-free, three-strand DNA assembly of all six junctions of a Hall magnetometer.

FIG. 4B shows an SEM micrograph at higher magnification of the three active Hall junctions, where the ratio of beads entirely or partially over the Hall junction versus beads outside of the junction are 8/41.

FIG. 4C shows an SEM micrograph at higher magnification of the three active Hall junctions, where the ratio of beads entirely or partially over the Hall junction versus beads outside of the junction are 11/68.

FIG. 4D shows an SEM micrograph at higher magnification of the three active Hall junctions, where the ratio of beads entirely or partially over the Hall junction versus beads outside of the junction are 12/73.

FIG. 4E depicts Hall responses for three active junctions (i, ii, iii) and three non-active control junction (ic, iic, iiic) are plotted as Hall voltage versus time.

FIG. 5A depicts native polyacrylamide gel electrophoresis characterization for a three-strand DNA assembly in the absence of nanoheads.

FIG. 5B depicts native polyacrylamide gel electrophoresis characterization for the three-strand DNA assembly in the absence of nanobeads.

FIG. 6A depicts a three-strand DNA assembly onto a mimic array (patterned on a GaAs substrate) for complementary target.

FIG. 6B depicts a three-strand DNA assembly onto a mimic array (patterned on a GaAs substrate) for non-complementary target.

### DETAILED DESCRIPTION

The systems, devices, and methods described herein have a wide number of applications and uses. For example, the devices and methods described herein may be used to detect nucleic acid sequences present in high copy number (e.g., infectious disease vectors, bioweapons, clinical assays of tissue samples for some genomic mutations). The devices and methods described herein may also be used to detect nucleic acid sequences present in small copy number, and distinguish nucleic acid sequences that arc highly similar (e.g., clinical assays of small tissue samples for genomic mutations, including point mutations; forensic analysis; biomedical research analysis, etc.).

The systems, devices, and methods described herein may also be used to detect biomolecular interactions for biomolecules other than nucleic acids (e.g., protein analysis for clinical assays or biomedical research), and potentially larger and more complex, soluble structures. In some instances, label-free analyses may be restricted to biomolecules that have at least two independent recognition sites. For example, proteins that contain two distinct epitopes that independently and simultaneously bind separate antibodies.

In one aspect, this disclosure generally relates to systems, devices, and methods that use micro-Hall magnetometry to detect interactions between two or three biological molecules. In embodiments, the devices detect sequence-specific DNA hybridization. In certain embodiments, the devices are capable of two-stranded (not “label-free”) detection of specific DNA sequences. In these embodiments, the target strand is labeled with a magnetic bead to provide an electrical Hall signal.

In certain embodiments, the devices or systems are capable of three-stranded (“label-free”) detection of specific DNA sequences. In these embodiments, there is no requirement for covalent modification of the target DNA sequence of interest. Typically, complementary DNA sequences that capture the magnetic target sequence are associated with the micro-Hall magnetometer (complimentary to part of the target sequence) and a second strand that may be associated with a small, paramagnetic bead (complimentary to another, distinct part of the target sequence). When all three strands are present, the target strand, in certain embodiments, serves to operably couple the magnetic bead to the micro-Hall magnetometer, and an electrical signal provides a signal related to its presence.

### Hall Device

Generally, the Hall devices or No-sensors described herein may be any magnetic sensing device with Hall effect sensors, e.g., a transducer that varies its output voltage in response to a magnetic field. In some embodiments, the Hall effect sensors respond to an applied magnetic field by producing a proportional voltage.

In embodiments, the Hall device comprises a plurality Hall junctions. In other embodiments, the device comprises hundreds to thousands of Hall junctions. In still other embodiments, the device comprises more than 100 Hall junctions. In certain embodiments, the Hall device comprises between 2 and 20 Hall junctions. Devices and systems with more Hall junctions are envisioned.

Generally, the Hall junctions may be of any size that allows for the adequate detection of biomolecular interactions. In embodiments, the Hall junctions have an area of from about 0.5 to about 2 μm². In some embodiments, the Hall junctions have an area of from about 0.7 to about 1.5 μm². In further embodiments, the Hall junctions have an area of from about 0.8 to about 1.2 μm². In still further embodiments, the Hall junctions have an area of about 1 μm².

Generally, the Hall junctions can be mass produced using standard photolithography and fabrication methods, and can operate at high frequency and field allowing for phase sensitive detection of the transient fields associated with magnetic beads, including, for example, SPM nanoscale beads. As a result, Hall bio-sensors may offer a label-free alternative to exclusively fluorescence based microarray technologies due to their ability to provide electrical readout, operate at room temperature, obtain single bead sensitivity, and be fabricated down to the nanoscale, while remaining biocompatible.

In embodiments, the sensitivity of the Hall devices or bio-sensors may be adjusted by adjusting the properties of the one or more Hall junctions. In one embodiment, the sensitivity is adjusted by changing the size of the Hall junction, the frequency of the ac field oscillation, the moment of the magnetic bead, or a combination thereof.

In embodiments, the devices described herein permit about 5 ppm to about 100 ppm detection in a 25 μL droplet of a biological sample. In some embodiments, the device described herein permit about 5 ppm to about 80 ppm detection in a 25 _{[}μL droplet of a biological sample. In other embodiments, the device described herein permit about 5 ppm to about 60 ppm detection in a 25 μL droplet of a biological sample. In further embodiments, the device described herein permit about 5 ppm to about 40 ppm detection in a 25 μL droplet of a biological sample. In still further embodiments, the device described herein permit about 5 ppm to about 20 ppm detection in a 25 μL droplet of a biological sample. In certain embodiments, these adjustments can permit about 1.0 ppm detection in a 25 μL droplet of a biological sample when the three-strand detection method described herein is used.

In embodiments, the systems or devices described herein are contacted with a solution comprising one or more target DNA strands. In some embodiments, the solution is a droplet. In other embodiments, the systems or devices described herein are submerged in a solution comprising one or more target DNA strands.

In embodiments, the platform may be capable of discriminating target DNA at <10 ppm in the presence of extraneous DNA and has been shown to operate at the single DNA per nanobead detection level. The sensitivity of the Hall device or bio-sensor may offer great potential for label-free nucleic acid detection at the genomic level, which may be integrated into a more complex POC device through further micro-fabrication.

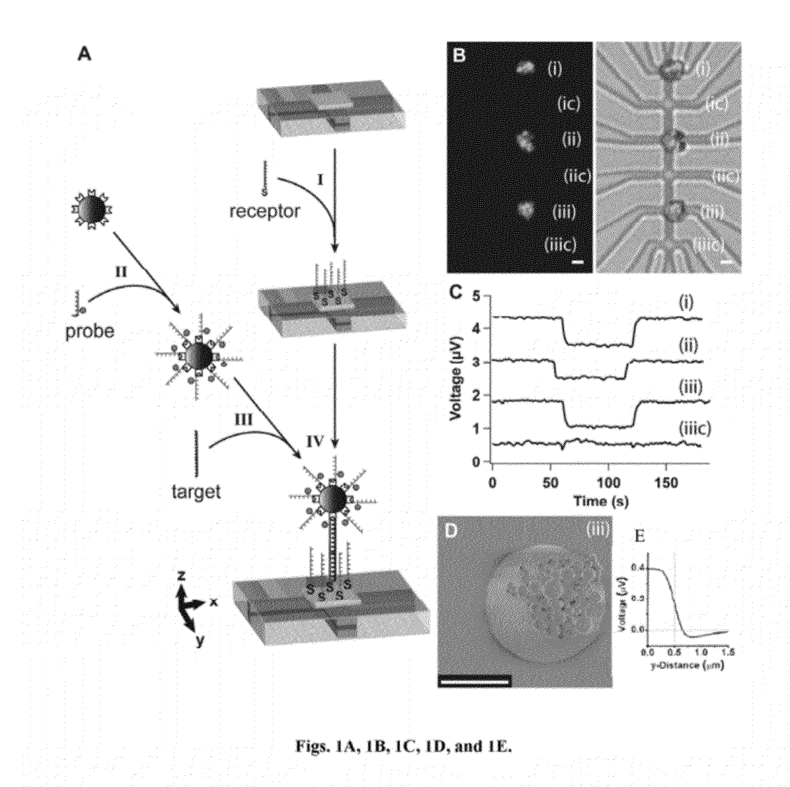

A schematic of the Hall bio-sensor for label-free detection of a ssDNA sequence by three-strand annealing, according to one embodiment described herein, is shown in FIG. 1A. Generally, the label-free target DNA may be detected by immobilization at the Hall device via complementary base pairing with receptor DNA pre-assembled on the Hall device surface to additional complementary probe DNA with an internal fluorescent marker pre-conjugated to the surface of a magnetic nanobead resulting in a detectable Hall signal. In other words, the magnetic bead is coupled to the Hall bio-sensor due to the interactions between the target DNA strands, the receptor DNA strands, and the probe DNA strands. The nanobead in this depiction is not drawn to scale.

In certain embodiments, the Hall device may be composed of six 1 μm² Hall junctions (FIG. 1B) etched into an epitaxially grown, vertically integrated InAs quantum well heterostructure (see, e.g., Mihajlovic, G. et al., APPLIED PHYSICS LETTERs 87, 2007; Mihajlovic, G., et al., J. OF APP. PHYS. 102, 2007; Manandhar, P. et al. NANOTECHNOLOGY 20, 2009). FIG. 1B illustrates the optical microscopy characterization (wide-field fluorescence and DIC overlay) of the three-strand DNA assembly of this embodiment. The DNA assembly may be shown by fluorescence, including green fluorescence, which indicates the presence of probe DNA. The scale bar in the figure equals 2 μm.

In embodiments, the one or more Hall junctions may be isolated from the surrounding environment by any suitable means. In one embodiment, a layer of a suitable material is used to isolate the Hall junctions. In certain embodiments, the layer may have a thickness of about 20 nm to about 100 nm. In other embodiments, the layer may have a thickness of about 40 nm to about 80 nm. In still other embodiments, the layer may have a thickness of about 60 nm. In particular embodiments, the micro-fabricated Hall junction is isolated from the surrounding environment by a 60 nm layer of silicon dioxide. In further embodiments, the layer may be treated or modified with another substance to minimize unwanted effects, such as, for example, bio-fouling. To minimize bio-fouling of the device by the biological constituents in the sample via non-specific interactions, the exposed SiO₂ surface may be modified by a polyethylene glycol conjugated silane moiety, such as, for example, a 2-[methoxy(polyethylene)propyl]trimethoxy silane moiety (Kannan, B. et al., NANO LETTERS 4, 2004, 1521-1524).

In certain embodiments, as shown in FIG. 1B, the Hall device or biosensor has six Hall junctions that are divided into a set of three bio-active sensors (i, ii, iii) and three non-active controls (ic, iic, iiic) via patterning only over the bio-active sensors with 3 μm×3 μm gold pads evaporated onto the SiO₂ layer. In these embodiments, the bonding pads provide a site for self-assembly of the receptor single strand DNA onto the surface of the Hall junction withouth modifying the properties of the InAs quantum well heterostructures; the non-active controls do not have the gold bonding pad.

Specificity, in some embodiments, may be directly related to fidelity of annealing of the target sequence to the probe and receptor strands. The use of dual modality detection may be advantageous for verification of biological assembly, while the use of three active and three non-active junctions provides statistical validation of the presence of the target sequence. In certain embodiments, the use of a SPM nanobead does not hinder specificity of W-C base pairing for the target nucleic acid as evidenced by sequence-specific DNA hybridization.

### Magnetic Bead

Generally, the magnetic bead may comprise any paramagnetic or superparamagnetic (SPM) material, and may be of any shape. Although the term “bead” is used throughout this disclosure, the term should not be construed as implying that the magnetic beads are spherical or substantially spherical. In embodiments, the bead may be shaped substantially like a sphere, cube, frustum, pyramic, prism, torus, tetrahedron, hexahedron, octahedron, dodecahedron, icosahedron, etc. Combinations of one or more of these shapes also may be used.

In embodiments, the magnetic bead is a nanoscale magnetic bead. In embodiments, the nanoscale magnetic beads have a mean size of from about 100 to about 700 nm. In some embodiments, the nanoscale magnetic beads have a mean size of from about 200 to about 500 nm. In other embodiments, the nanoscale magnetic beads have a mean size of from about 300 to about 400 nm. In certain embodiments, the nanoscale magnetic beads have a mean size of about 350 nm.

In embodiments, the probe DNA strands may be pre-conjugated to the magnetic bead any any means known in the art. In one embodiment, the probe DNA is pre-conjugated to the magnetic bead through a biotin-streptavidin linkage.

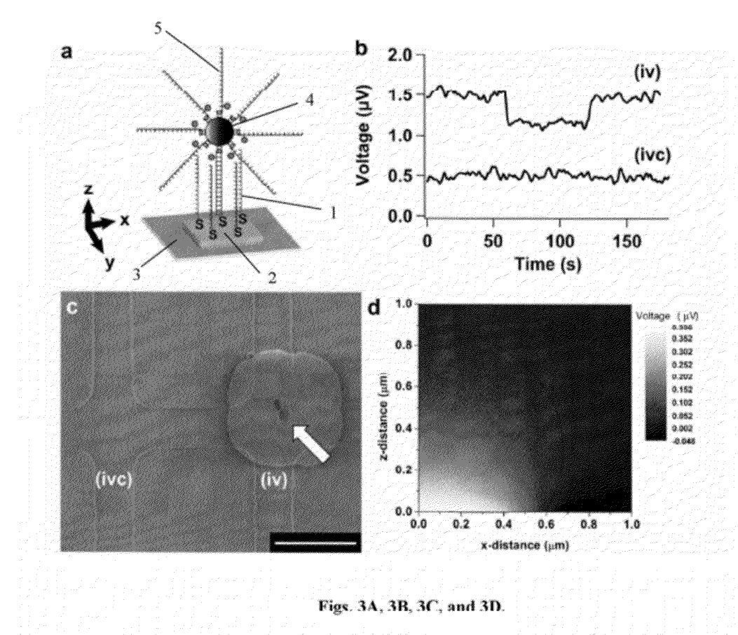

In certain embodiments, an SPM bead improves sensitivity of detection, since an applied de-field allows background discrimination, while the ac-magnetic field allows for oscillation of the magnetic moment in the SPM bead resulting in phase sensitive detection. In particular embodiments, the Hall magnetometer response drops off quickly at the edge of the Hall junction, an important characteristic for complex multiplexing applications. At the frequency detection employed in one embodiment, the 3-D plot in FIG. 3D indicates sensitivity towards longer DNA or more complex architectures is achievable even up to distances approaching 0.9 μm. Not wishing to be bound by any particular theory, it is believed that higher frequency measurements will decrease the noise levels and therefore increase the sensitivity of the device to the magnetic bead position. It is also believed that reduction of the dimension of the Au pad and improved registry, as well as bead homogeneity, will improve the overall device performance allowing Hall magnetometry to improve POC diagnostics.

### Microarray

Generally, the devices described herein may be incorporated into a microarray. In some embodiments, the devices described herein may be used in a POC apparatus.

Generally, extrapolation of the devices to a microarray of selectively labeled Hall sensors can provide a highly useful bio-sensor platform. Not wishing to be bound by any particular theory, it is believed that such a microarray might eliminate concerns associated with sample amplification (Ince, J. et al., EXPERT REVIEW OF MEDICAL DEVICES 6, 2009, 641-651), and that such an array may allow rapid screening for nucleic acid targets of biomedical interest such as pathogens, or disease-related mutations (Parmacek, M. S. et al., PROGRESS IN CARDIOVASCULAR DISEASES 47, 2004, 159-176; Kohler, et al., PHYSIOLOGICAL GENOMICS 14, 2003, 1 17-128).

### Method for Three-Strand Detection

In embodiments, the methods for three-strand detection described herein may be performed without altering the target DNA strands. Typically, the method for three-strand detection comprises pre-assembling one or more receptor DNA strands on a surface of a Hall device, pre-conjugating one or more probe DNA strands to a magnetic bead, and detecting the coupling of the magnetic bead to the Hall device. In these methods, the one or more receptor DNA strands and the one or more probe DNA strands are configured to interact with one or more target DNA strands via complementary base pairing.

In certain embodiments, the Hall device or bio-sensor platform may be assembled in parallel steps in order to limit the processing time at the point of target detection. In embodiments, the first step is the self-assembly of receptor DNA strands on the Hall device or sensor. In one embodiment, this step requires the self assembly of the receptor ssDNA onto the bio-active Au pads at room temperature for 6 hrs (FIG. 1A-I).

In embodiments, the probe DNA, which may be labeled with an appropriate dye, is then pre-conjugated to a magnetic bead. In certain embodiments, the dye (fluorescein dT) labeled probe ssDNA (red) may be pre-conjugated to SPM beads (mean size 350 nm, Bangs Laboratories) in a separate step at 303 K for 1 hr (FIG. 1A-II).

In embodiments, a target DNA strand may be detected by detecting the coupling of the magnetic bead to the Hall device. In one embodiment, the coupling of the magnetic bead to the Hall device may be detected by annealing the probe DNA strand to the target DNA, then annealing the receptor DNA strand to the target DNA, and detecting the voltage response in the Hall device. In other embodiments, the coupling of the magnetic bead to the Hall device may be detected by annealing the receptor DNA strand to the target DNA, then annealing the probe DNA strand to the target DNA, and detecting the voltage response in the Hall device. In some embodiments, the coupling of the magnetic bead to the Hall device may be detected by simultaneously annealing the receptor DNA strand and probe DNA strand to the target DNA strand, and then detecting the voltage response in the Hall device.

In the devices described herein, the target, probe, and receptor DNA strands interact with each other due to complementary base pairing, which is well known in the art. In some embodiments, complementary base pairing enables the annealing steps described herein to occur. Complementary base pairing, in some embodiments, relies on Watson-Crick (W-C) base pairing.

In one embodiment, target DNA detection is accomplished through a label-free step by first annealing the probe strand to the target strand at 353 K for 2 min and allowed to cool slowly to room temperature over 1 hr (FIG. 1A-III), followed by annealing of the target assembly with the receptor sequence at the platform surface at room temperature for 2 hrs (FIG. 1A-IV). Unbound nucleic acids may be removed by magnetic separation prior to tethering the SPM bead to the Hall device. In certain embodiments, the parallel steps are carried out simultaneously over the Hall platform. In certain embodiments, the parallel steps are carried out in stepwise fashion. In the embodiment shown in FIG. 1, the probe strand and the target strand can be pre-hybridized prior to the detection of the target DNA sequence.

Although the parallel steps may be carried out simultaneously over the Hall platform, stepwise assembly may allow a convenient amplification step for the target ssDNA from a mixture containing extraneous DNA fragments using magnetic head sorting. In addition, various DNA sequences may be simultaneously detected in the biological matrix since each magnetic bead and probe strand can be bar-coded (Li, Y. G. et ai., NATURE BIOTECHNOLOGY 23, 2005, 885-889) (PL, SPM bead moment), and may contain a unique DNA sequence and the receptor DNA on the Hall junction may be selectively dip-penned for multi-sequence analysis (Demers. L. M. et al., SCIENCE 296, 2002, 1836-1838).

In the devices described herein, the probe and receptor DNA strands, or the receptor strands in the two-strand devices interact with each other in a way that couples the magnetic bead to the Hall device of bio-sensor. In one embodiment, the coupling of the magnetic bead to the Hall device immobilizes the magnetic bead about the Hall device. In another embodiment, the coupling of the magnetic head to the Hall device localizes the magnetic bead over a Hall junction. In these embodiments, the sensitivity of the Hall junction may be increased so that its active area responds only to the coupled magnetic bead.

In one embodiment, label-free discrimination is shown for a 35 base pair (bp) single strand (ss) DNA target as a mimic for pathogenic DNA detection in a biological matrix using Hall magnetometry. The label-free detection strategy, in this embodiment, utilizes Watson-Crick (W-C) base pairing of three ssDNA sequences (target, probe, receptor) to localize a super paramagnetic (SPM) 350 nm. bead over the micro-fabricated 1 μm² Hall junction resulting in a local magnetic field that induces a subsequent voltage response in the device. Since, in this embodiment, the change in voltage will only occur for a SPM bead positioned directly over the active Hall junction and will not significantly respond to the presence of magnetic beads outside of the active area (Li, Y. G. et al, APPLIED PHYSICS LETTERS 80, 2002, 4644-4646), the device may be further developed into a microarray.

The results for 35-bp target DNA detection are shown both optically and electrically for the Hall sensor after drying (FIG. 1B-1C). In this embodiment, three-strand DNA assembly on the Hall bio-sensor was accomplished at 298 K using a 25 μL droplet (7 μM DNA) of target DNA pre-conjugated to magnetic nanobeads. As shown in FIG. 1B, assembly of the three-strand DNA complex onto an Au pad (grey circle) over a Hall junction, in this embodiment, is clearly observed using wide-field fluorescence overlaid with differential interference contrast (DIC) microscopy (FIG. 1B), where the observed green photoluminescence corresponds to the fluorescein labeled probe strand.

For the detection of DNA annealing, in some embodiments, the presence of the SPM bead is measured as a change in voltage by the use of both ac and dc magnetic fields. The use of both ac and dc fields, in these embodiments, allows for a binding event signal to be cleanly isolated by using lock-in detection. In the absence of the external dc field, in these embodiments, no signal is detectable in the Hall junction.

The voltage responses of the Hall bio-sensor's three active junctions and a single control junction, for this particular embodiment, are shown in FIG. 1C. FIG. 1C shows the Hall responses for three active junctions (i, ii, iii) and a single control junction (iiic), which are plotted with Hall voltage versus time. Not wishing to be bound by any particular theory, it is believed that the presence of nanobeads over the active Hall junctions resulted in a drop in Hall voltage when a dc magnetic field was applied. Consistent with the optical results, functionalized Hall junctions i, ii, and iii, in this embodiment, exhibited a sharp drop in Hall voltage during application of an external de magnetic field, and returned to baseline when the de magnetic. field was removed. The observed voltages and respective signal to noise (S/N) ratios for Hall junction i, ii, and iii, were 0.79 μV (S/N 39.5), 0.55 μV (S/N 27.5), and 0.78 μV (S/N 39), respectively. The noise level for the three control junctions (ic, iic, iic) was 0.02 μV (all data shown in SI). The Hall sensor was operated in constant current mode with an applied dc current of 50 μA. An ac magnetic field of 3.76 mT at 93 Hz induced magnetization of the nanobeads. A 70.6 mT dc magnetic field (NdFeB) was applied perpendicular to the Hall junction to shift the magnetization of the nanobeads to lower susceptibility as given by ΔV_{H} ≧ΔM where ΔM is the change in the ac magnetization before and after the dc field was applied. The dual use of both ac and dc fields allowed for the appropriate signals to be cleanly isolated utilizing lock-in detection.

FIG. 18 shows that the registry of the Au pad and the underlying Hall junction resulted in a majority of beads lying outside the junction area in this particular embodiment, and a smaller number directly over the Hall junction (FIG. 1D). FIG. 1D depicts an SEM image used to evaluate the location and quantify the number of nanobeads contributing to the Hall response for (iii). FIG. 1E shows the theoretical device signal stemming from a single 344 nm SPM bead as a function of position over the Hall junction, further illustrating the local sensitivity of Hall magnetometry. The scale bar in the figure equals 2 ρm.

Regardless, the observed voltage response for the three pads was similar in this particular embodiment. In FIG. 1C, the observed voltage response was therefore dependent on the particle position relative to the Hall junction center as extrapolated from experimental data in FIG. 1E for a 344 nm magnetic bead approximately 272 nm from the Hall device surface. The largest voltage change may be expected for beads directly over the center of the Hall junction, falling rapidly for beads positioned >0.5 μm from the device center. The calculated voltage response, for this particular embodiment, indicated nanobeads that lie outside of the junction will not significantly contribute to the observed Hall response due to the stray magnetic field being out of range of the underlying quantum well layer.

In embodiments, an independent optical signature from a molecular dye appended to the synthetic probe DNA provides a redundant optical response. The molecular dyes may be used in both the two-strand and three-strand methods described herein.

In certain embodiments, the devices described herein may utilize a fluorescent label on the DNA strands associated with the magnetic beads. The fluorescent label may be used as a redundant signal during the development of the devices and methods described herein because the fluorescent signal correlates with the magnetic signal—it is not, however, essential for micro-Hall magnetic detection.

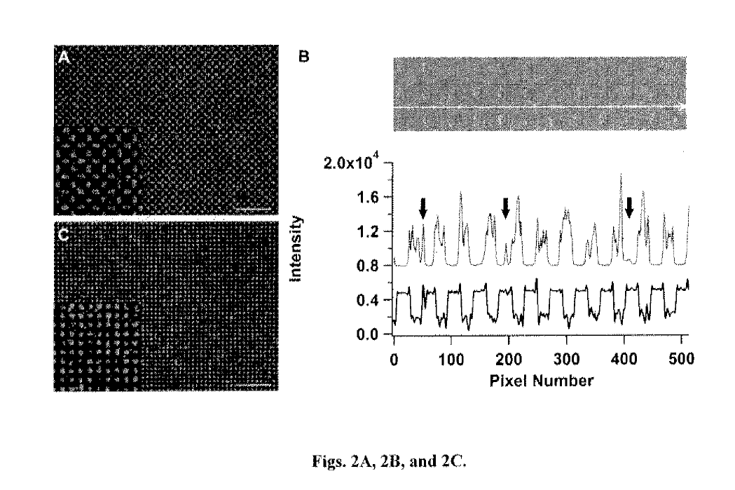

FIG. 2 depicts another embodiment of the device. FIG. 2A depicts a three-strand DNA assembly on a mimic array (patterned on a GaAs substrate) for complementary target only. The inlay in the lower portion of FIG. 2A shows an enlarged portion of the depiction. The scale bar equals 50 μm. FIG. 213 depicts a line scan of the wide-field fluorescence microscopy image in FIG. 2A—showing fluorescein-labeled probe DNA (top) and DIC (bottom) intensity correlates fluorescence intensity with nanobeads located primarily over gold pads, where the black arrows signify the presence of a small number of non-specifically bound nanobeads.

In the embodiment shown in FIG. 2, the optical microscopy image reveals partial assembly of beads onto the pad, although the beads are not optically resolvable at the magnification used (40× objective). The lack of green emission (represented by the dark rectangles in FIG. 2B) outside of the Au pad region confirms the absence of non-specific binding elsewhere on the device. Experiments using optical mimics for the Hall device on 2×4 μm patterned GaAs indicated that the level of intensity discrimination is >10,000 counts above background for selective target DNA binding at the Au pads in buffered solution (FIG. 2A). The line scan of the wide field microscopy image (FIG. 2B) indicated green fluorescence (represented by the dark rectangles) intensity was observed only over the Au pads with little significant intensity over the control PEGylated regions surrounding Au pads (identified with black arrows). Not wishing to be bound by any particular theory, it is believed that the signal fluctuations did not indicate single bead response as the fluorescein intensity depends on the particle size, number of DNA, labeling efficiency, and focal plane of the microscopy image.

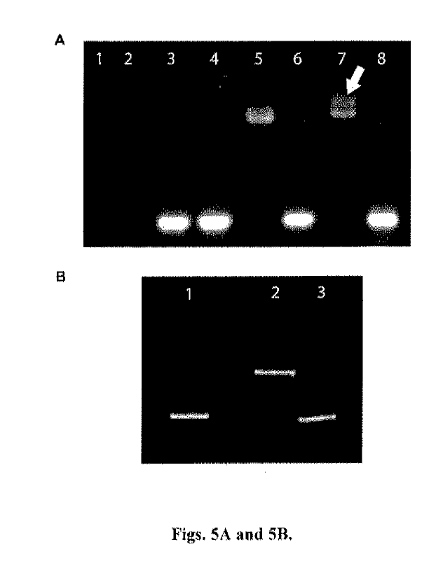

Evidence for three-strand DNA assembly was experimentally verified using a gel shift assay (FIGS. 5A and 5B). The number of DNA strands per bead was estimated by considering the mean size and binding capacity of the commercially available magnetic nanobeads, wherein each 350 nm bead contains approximately 36,000 probe dye labeled DNA strands.

FIGS. 5A and 5B further depict one of the embodiments described herein. FIG. 5A depicts native polyacrylamide gel electrophoresis characterization for the three-strand DNA assembly in the absence of nanobeads showing different mobilities of (1) visual loading dye not observed under fluorescent excitation, (2) reporter strand (disulfide 20mer) and complementary target, (3) probe strand (fluorescein labeled 15mer), (4) reporter and probe strands, (5) probe and complementary target, (6) probe and non-complementary target, (7) reporter, probe, and complementary target, (8) reporter, probe, and non-complementary target. Not wishing to be bound by any particular theory, it is believed that the visible bands (3-8) originated from the internal fluorescein modification on the probe strand and the differences in mobility reflect the assembling of the three-strand DNA structure. The arrow in (7) signifies the three-strand ‘DNA product. FIG. 5B depicts native polyacrylamide gel electrophoresis characterization for the three-strand DNA assembly in the absence of nanobeads showing different mobilities of (1) probe strand (fluorescein labeled 35mer), (2) complementary reporter and probe strands, (3) non-complementary reporter and probe strands. Not wishing to be bound by any particular theory, it is believed that the visible bands (1-3) originated from the internal fluorescein modification on the probe strand and the differences in mobility reflect the assembling of the two-strand DNA structure.

The scanning electron microscopy (SEM) data for junction iii (FIG. 41)) of the above-described embodiment indicated ˜73 beads are present on the gold pad (3×3 μm) in which ˜12 nanobeads directly or partially overlap the outline of the Hall junction and the remaining beads lie within 2 μm from the center of the junction. The result indicated that, in this embodiment, the measured voltage is the sum of all beads on the Au pad, with the most significant contribution of the signal arising from 12 beads directly over the center of the Hall junction (southeast corner of the pad in FIG. 1D). Not wishing to be bound by any particular theory, it is believed that the observed signal originating from a maximum of 9 nanobeads (344 nm) arranged in a single layer packed over the entire Hall junction is predicted to have an electrical readout of 2.98 μV. Since the nanobeads of this particular embodiment have a wide size dispersion and thus iron content, an exact correlation may not exist; however, an empirical correlation can be made by comparing the observed voltage to the number of beads over the active Hall junction.

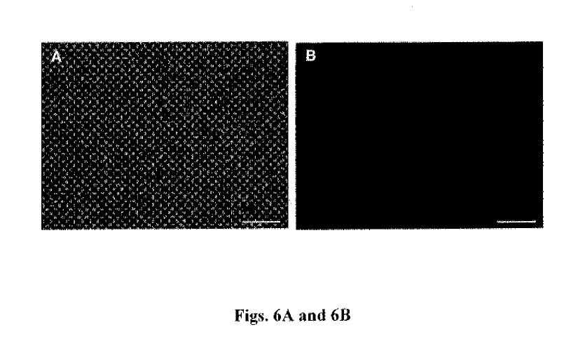

For this embodiment, fidelity of the three-strand detection strategy is demonstrated by the ability to discriminate target ssDNA in the presence of extraneous (non-complementary) sequences in solution, particularly at low levels of target DNA. The ability to discriminate target DNA in the presence of non-target sequences was analyzed by optical microscopy on 3 μm patterned GaAs Hall device mimics in a buffered solution (FIG. 2C). The presence of fluorescently tagged nanobeads was observed at a concentration of 36 pM at 10 ppm level of target to non-target sequences, which corresponds statistically to less than a single complementary target DNA sequence per 350 nm nanobead (FIG. 2C). FIG. 2C depicts a three-strand DNA assembly on a mimic array (patterned on a GaAs substrate) for a 10 ppm target in non-target DNA. The inlay in the lower portion of FIG. 2C shows an enlarged portion of the depiction. The scale bar equals 50 μm. Comparison to a mimic array in which the receptor strand is non complementary to the target strand indicates no significant nonspecific DNA binding occurs (FIGS. 6A and 6B).

FIG. 6 depicts a particular embodiment of the device described herein. FIG. 6A depicts a three-strand DNA assembly onto a mimic array (patternred on a GaAs substrate) for complementary target. FIG. 6B depicts a three-strand DNA assembly onto a mimic array (patterned on a GaAs substrate) for non-complementary target. The presence of green fluorescence (shown as white spots in FIG. 6A) indicates the location of the SPM nanobeads. Scale bars equals 50 μm.

Although the ability to detect target DNA at ppm levels is demonstrated in FIG. 2, the dynamic range achievable is also important for certain embodiments of the devices described herein. Based on the previously-described magnitude of the voltage response for ˜12 beads over the Hall junction in a particular embodiment, single bead detection should be achievable, at least to a certain extent.

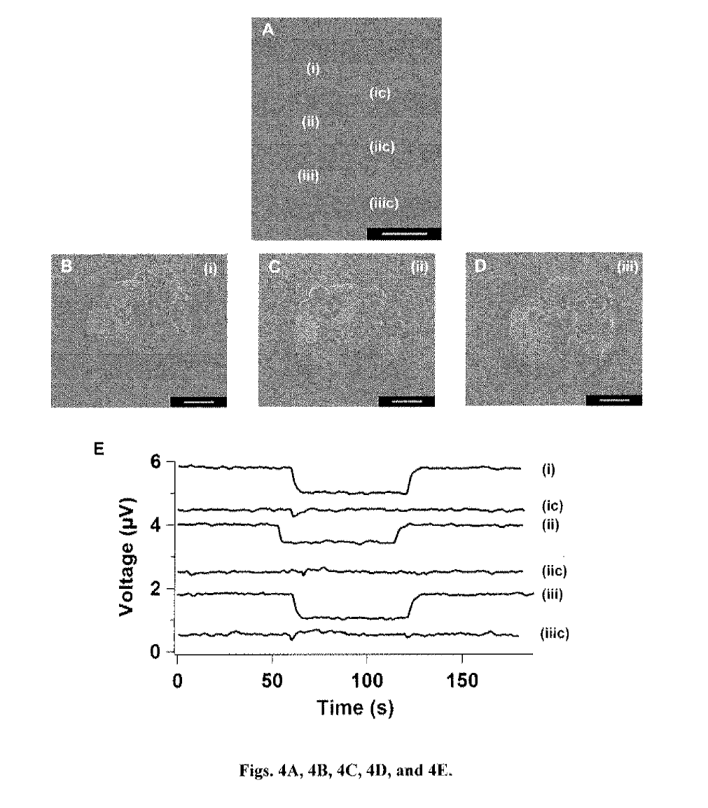

FIG. 4 further depicts one of the embodiments described herein. FIG. 4A shows an SEM micrograph depicting label-free, three-strand DNA assembly of all six junctions of a Hall magnetometer. Scale bar of FIG. 4A equals 10 μm. FIGS. 48-4D depict SEM micrographs at higher magnification of the three active Hall junctions, where the ratio of beads entirely or partially over the Hall junction versus beads outside of the junction are FIGS. 4B— 8/41, FIG. 4C— 11/68, and FIG. 4D— 12/73. Scale bars FIG. 4B-4D equal 1 μm. FIG. 4E depicts Hall responses for three active junctions (i, ii, iii) and three non-active control junctions (ic, iic, iiic) plotted as Hall voltage versus time; the presence of SPM nanobeads over the active Hall junctions results in a drop in Hall voltage when a dc magnetic field is applied. When the de magnetic field is removed the signal returns to baseline.

### Methods for Two-Strand Target Detection

Other methods are provided herein comprising pre-conjugating one or more target DNA strands to a magnetic bead, pre-assembling one or more receptor DNA strands on a surface of a Hall device, and detecting the coupling of the magnetic bead to the Hall device. In these methods, the one or more receptor DNA strands are configured to interact with the one or more target DNA strands via complementary base pairing.

Several of the previously-described techniques for three-strand target detection are applicable to two-strand target detection, and may be used where appropriate. In certain embodiments, the devices described herein are able to identify a single bead hound to target DNA (35 bases) and is amenable to the discrimination of DNA at the 364 pM concentration in a background of 36 μM non-complementary DNA (<10 ppm).

One embodiment of the method for two-strand target detection is depicted in FIG. 3. In FIG. 3A, one or more acceptor DNA strands I are assembled on a Hall junction 2 of a Hall device 3. The acceptor DNA strands 1 interact, due to complementary base pairing, with the target DNA strands 5, which have been conjugated to a magnetic bead 4. As a result, the magnetic bead 4 is coupled to the :Hall device 3.

In FIG. 3, the Hall voltage response for one embodiment of the 2-strand target detection method (FIG. 3A) is shown. FIG. 3 generally depicts one embodiment of a sequence-specific two-strand DNA assembly and subsequent Hall detection of a single 344 nm nanobead. In this embodiment, the voltage response (FIG. 3B) and SEM image (FIG. 3C) from two beads binding on the Au pad indicated a response of 0.34 μV (0.044 μV background) following DNA annealing. FIG. 3B depicts, for this embodiment, Hall response data for the active (iv) and control Hall junction (ivc) plotted as Hall voltage versus time, where the drop in Hall voltage corresponds to the presence of a magnetic nanobead. FIG. 3C depicts an SEM image which confirms only one nanobead contributed significantly, in this embodiment, to the signal measured in (iv). The scale bar equals 2 μm.

In this embodiment, the Hall voltage is likely due to just one of the two beads—the bead that lies directly over the Hall junction (arrow in FIG. 3C). Analogous to FIGS. 1D and 1E, the single bead assumption is easily confirmed, in this embodiment, by plotting the voltage of the Hall device as a function of SPM bead position relative to the center of the Hall junction: the darker shading inside the circle, which is the outline of the noise floor, indicates a SPM particle positioned at the center of the device, while the darker shading outside the circle a SPM outside the detectable range of the Hall junction (FIG. 3D). In other words, the darker shading inside the circle represents the high end of the accompanying voltage scale, while the darker shading outside the circle represent the low end of the accompanying voltage scale. More specifically, FIG. 3D depicts, for the above-described embodiment, the theoretical Hall device cross-sectional response for a single 344 nm SPM bead as a function of position from the center of the junction. The darker shading inside the circle indicates the strongest change in voltage, the lighter shading in the figure indicates the weakest voltage change, and the darker shading outside the circle indicates a negative voltage readout.

As described herein, efficient, label-free (or non-label-free) target detection can be realized in a POC device through the integration of biology and nanotechnology by capitalizing on the high biological specificity of DNA base pairing, the scalability of nanotechnology, the selectivity of self assembled monolayer technology (Wink, T. et al., ANALYST 122, 1997, R43-R50; Herne, T. M. et al., J. OF AM. CHEM. SOC. 119, 1997, 8916-8920), and the sensitivity of micro-fabricated electronics.

The devices and methods described herein may be used in various arrays of detectors which are capable of identifying multiple target molecules in a single sample.

Modifications and variations of the methods and devices described herein will be obvious to those skilled in the art from. the foregoing detailed description. Such modifications and variations are intended to come within the scope of the appended claims.

### EXAMPLES

### Example 1

### Substrate Fabrication and Passivation

A 1 μm² Hall junction was fabricated as a vertically integrated system using a combination of photolithography and wet chemical etching techniques. Following etching of the Hall junction into the surface of an epitaxially grown heterostructure consisting of a GaAs substrate containing an InAs quantum well core, SiO₂ (60 nm) was sputtered onto the device followed by a layer of Ti (5 nm), and deposition of 3 μm Au pads (20 nm thick) directly over the protected Hall junction. Registry of the Au pad was accomplished by photolithography using alignment markers in the photomask. Mimic microarrays were fabricated onto the <100> face of a single crystal GaAs wafer using a similar protocol.

All substrates were cleaned prior to use for 1 min at low power in oxygen plasma (Harrick Plasma PDC-001). The substrates were rinsed with absolute ethanol for 1 min and dried under a constant stream of nitrogen gas. The polyethylene glycol silane solution was prepared in a freshly cleaned and dried glass graduated cylinder containing 100 μL of 2-[Methoxy(Polyethyleneoxy)Propyl]-Trimethoxysilane (Gelest) and 40 μL of concentrated hydrochloric acid in 50 mL of HPLC grade toluene and sonicated for 10 min prior to use. The pre-cleaned substrates were immersed in the freshly prepared polyethylene glycol silane solution for 1 hr. The samples were removed and rinsed in a vial containing 10 mL of toluene followed by another 10 mL, of toluene and sonicated for 2 min. Finally, the samples were removed and rinsed in 10 mL of 18 MΩ nanopure H₂O before drying under a constant stream of nitrogen gas. The dried samples were placed in an oven at 115° C. for 1 hr.

### Example 2

### DNA Immobilization and Hybridization (2-Strand)

Synthetic DNA oligonucleotides were commercially synthesized (Midland Certified

Reagent Company) and consisted of a 5′ disulfide modified complementary receptor sequence 5′-RSSR-GAC TAC TCT ATC GGC AGC TAA GAT TGT CAC AGT CG-3′, a 5′ disulfide modified non-complementary receptor sequence 5′-R.SSR-CGA CTG TGA CAA TCT TAG CTG CCG ATA GAG TAG TC -3′, and a 5′ modified biotinlyted probe sequence with an internal fluorescein dT 5′-Biotin-CGA C/iFLUORdT/G TGA CAA TCT TAG CTG CCG ATA GAG TAG TC-3′. The lyophilized DNA was buffer exchanged using a NAP-V size exclusion column (GE H ealthcare) equilibrated with 20 mM sodium phosphate buffer, 50 mM NaCl pH 7.0.

The receptor DNA (not previously reduced) was incubated on top of the device for 6-6.5 hrs in an enclosed incubation chamber containing a supersatured NaCl solution in the form of a 50 μL droplet at a DNA concentration of 9 μM. The RSSR disulfide functionality was not reduced prior to assembly on the device. The device was then immersed in 5 mL of 18 MΩ nanopure H₂O containing 0.1% Teen-20 (v/v) and twice in 5 mL of 18 MΩ nanopure H₂O to rinse and remove unbound sequences of DNA. The device was then dried under a constant stream of nitrogen gas. The reporter DNA was bioconjugated to the superparamagnetic (SPM) nanobead (350 nm mean size, Bangs Laboratories) through a biotin-streptavidin linkage at 30° C. for 1 hr. The DNA-nanobead conjugate was purified away from free DNA using magnetic separation and washing the sample 5 times with 20 mM sodium phosphate buffer, 300 mM NaCl pH 7.0. The hybridization assay was carried out by incubating a 25 μL droplet of target biotinlyted DNA (7 μM) bound to streptavidin coated SPM beads for 2-2.5 hrs in an enclosed incubation chamber containing a super saturated NaC1 solution as described herein. The device was then washed once in 5 mL of 20 mM sodium phosphate buffer with 300 mM NaCl at pH 7.0 containing 0.1% Tween-20 (v/v) and twice in 5 mL of 20 mM sodium phosphate buffer with 300 mM NaCl at pH 7.0. The device was then stored in 5 mL of 20 mM phosphate buffer with 300 mM NaCl at pH 7.0 and protected from ambient light.

### Example 3

### DNA Immobilization and Hybridization (3-Strand)

Synthetic DNA oligonucleotides were commercially synthesized (Midland Certified Reagent Company). The 3-strand DNA hybridization assay consists of a probe sequence 5′-TCA TTC ACA CAC/iFLUORdT/CG/3BIO/-3′ labeled with an internal fluorescein dT and biotin, receptor sequence 5′-/RSSR/GTC TTG TCT CCT GTC AGC TA -3′ with a disulfide modifier, a 35 base pair unmodified target sequence 5′-CGA GTG TOT GAA TGA TAG CTG ACA GGA GAC AAG AC-3′, and a 35 base pair unmodified non-target control sequence 5′-GTC TAA GAG TGT CCTT GGC TAT GAT CCG TGA GTA TG-3′. The DNA was prepared and assembled analogously to the 2-strand DNA, with the exception that a solution of the target 35mer was first appended to the DNA-nanobead bioconjugate at 80° C. and allowed to slowly cool over 1 hr. An assay was then carried out as described herein.

### Example 4

### Microscopy and Hall Measurement

Fluorescent microscopy was carried out on an inverted Nikon TE2000-E2 Eclipse microscope (Nikon. Instruments Inc.) equipped with a Nikon CFI Plan Apochromat 40× objective (NA 0.95, 0.14 mm WD). Wide-field imaging of the substrates utilized an EXFO E-Cite illumination source, a FITC filter (Chroma). Images were acquired on a Photometrics Coolsnap HQ₂ CCD camera. Bright-field overlays utilized differential interference contrast (DIC) to observe the differences in the index of refraction of the samples. The data was analyzed using Nikon NIS Elements software. Scanning electron microscopy (SEM) was carried out on a FEl Nova 400 Nano SEM. and utilizing a through-the-lens (TED) detector. The substrates were not coated with a conducting metal before imaging. The SEM images were acquired using a 32 scan average.

The detection of pre-immobilized SPM beads was achieved by employing an ac phase-sensitive technique previously reported by Besse et al (Besse, P. A., Boero, G., Demierre, M., Pott, V. & Popovic. R. Detection of a single magnetic microbead using a miniaturized silicon Hall sensor. Applied Physics Letters 80, 4199-4201 (2002). The Hall device was, in some instances, biased with a de current I=50 μA. and the beads were magnetized with an ac magnetic field; lock-in detection of the ac Hall voltage occured at the magnetic field frequency. The application of an additional dc magnetic field reduced the SPM bead's susceptibility and thus the ac magnetic field generated by the beads. This produced a drop in the ac Hall voltage signal indicating the presence of the beads.

We claim:

1. A system for detecting biomolecular interactions, the system comprising:
a Hall device;
one or more receptor DNA strands pre-assembled on the Hall device surface;
a magnetic bead; and
one or more probe DNA strands pre-conjugated to the magnetic bead;
wherein the one or more receptor DNA strands and the one or more probe DNA strands are configured to interact with one or more target DNA strands via complementary base pairing.

2. The system of claim 1, wherein the one or more receptor DNA strands are configured to interact with a first section of the one or more target DNA strands, and the one or more probe DNA strands are configured to interact with a second section of the one or more target DNA strands.

3. The system of claim 1, wherein the one or more probe DNA strands comprises a dye.

4. The system of claim 1, wherein the Hall device surface comprises a plurality of Hall junctions to which the one or more receptor DNA strands are assembled.

5. The system of claim 4, wherein the Hall junctions have an average size of from about 0.5 to about 2 μm².

6. The system of claim 4, wherein the Hall junctions are isolated from the surrounding environment by a protective layer.

7. The system of claim 6, wherein the protective layer comprises silicon dioxide.

8. The system claim 1, wherein the magnetic head is a nanoscale bead.

9. The system of claim 1, wherein the magnetic bead is an SPM bead.

10. The system of claim 1, wherein the device permits about 5 ppm to about 100 ppm detection in a 25 μL droplet of a biological sample.

11. A method for the detection of biomolecular interactions, the method comprising:
providing a Hall device onto which one or more receptor DNA strands are coupled; and
detecting an interaction of one or more target DNA strands with the one more receptor DNA strands and one or more probe DNA strands, wherein the one or more probe DNA strands are coupled to a magnetic bead, and the one or more probe DNA strands and the one or more receptor DNA strands interact with the one or more target DNA strands via complementary base pairing.

12. The method of claim 11, wherein detecting the interaction comprises:
annealing the one or more probe DNA strands to the one or more target DNA strands;
annealing the one or more receptor DNA strands to the one or more target DNA strands; and
detecting the voltage response in the Hall device.

13. The method of claim 12, wherein the annealing steps occur sequentially.

14. The method of claim 12, wherein the annealing steps occur simultaneously.

15. A system for detecting biomolecular interactions, the system comprising: a Hall device;
one or more receptor DNA strands pre-assembled on the Hall device surface;
a magnetic bead; and
one or more target DNA strands pre-conjugated to the magnetic bead;
wherein the one or more receptor DNA strands are configured to interact with the one or more target DNA strands via complementary base pairing.

16. The system of claim 15, wherein the Hall device surface comprises a plurality of Hall junctions to which the one or more receptor DNA strands are assembled.

17. The system of claim 16, wherein the Hall junctions have an average size of from about 0.5 to about 2 μm².

18. The system of claim 16, wherein the Hall junctions are isolated from the surrounding environment by a protective layer

19. The system of claim 18, wherein the protective layer comprises silicon dioxide.

20. The system of claim 15, wherein the magnetic bead is a nanoscale bead.

21. The system of claim 15, wherein the magnetic bead is an SPM bead.

22. A method for the detection of biomolecular interactions, the method comprising:
providing a Hall device onto which one or more receptor DNA strands are coupled; and
detecting an interaction of one or more target DNA strands, which are coupled to a magnetic bead, with the one or more receptor DNA strands, wherein the one or more receptor DNA strands interact with the one or more target DNA strands via complementary base pairing.

23. The method of claim 23, wherein detecting the interaction comprises:
annealing the one or more receptor DNA strands to the one or more target DNA strands: and
detecting the voltage response in the Hall device.


SEQUENCE LISTING (7 sequences)

SEQ ID NO: 1 - DNA, 35 bp, Artificial Sequence
  Description of Artificial Sequence Synthetic oligonucleotide
gactactctatcggcagctaagattgtcacagtcg

SEQ ID NO: 2 - DNA, 35 bp, Artificial Sequence
  Description of Artificial Sequence Synthetic oligonucleotide
cgactgtgacaatcttagctgccgatagagtagtc

SEQ ID NO: 3 - DNA, 35 bp, Artificial Sequence; identical to SEQ ID NO: 2
  Description of Artificial Sequence Synthetic probe

SEQ ID NO: 4 - DNA, 15 bp, Artificial Sequence
  Description of Artificial Sequence Synthetic probe
tcattcacacactcg

SEQ ID NO: 5 - DNA, 20 bp, Artificial Sequence
  Description of Artificial Sequence Synthetic oligonucleotide
gtcttgtctcctgtcagcta

SEQ ID NO: 6 - DNA, 35 bp, Artificial Sequence
  Description of Artificial Sequence Synthetic oligonucleotide
cgagtgtgtgaatgatagctgacaggagacaagac

SEQ ID NO: 7 - DNA, 36 bp, Artificial Sequence
  Description of Artificial Sequence Synthetic oligonucleotide
gtctaagagtgtccttggctatgatccgtgagtatg